(19) United States
(12) Patent Application Publication
(10) Pub. No.: US 2009/0054833 A1
(43) Pub. Date: Feb. 26, 2009

(54) Portable power neti sinus irrigator

(76) Inventors: Hong Cui O'hare, Tucson, AZ (US); Douglas Lee Smith, Tucson, AZ (US)

Correspondence Address: Hong Cui O'hare, 6032 S. Nantac Rim Dr., Tucson, AZ, 85706, US

(21) Appl. No.: 11/895,014

(22) Filed: Aug. 23, 2007

(51) Int. Cl. A61M 3/02

(52) U.S. Cl. 604/73

(57) ABSTRACT

A power neti pot has a mini pump, batteries, a detachable electric cord and plug, an AC/DC switch in the bottom of the base and an on/off switch on the side of the base. There is a cup with a lid seated on top of the base. An outlet tube connected to the pump is connected to a tapered spout on top of the cup. The pump delivers the liquid in the cup through the tube, exiting the spout into the sinus cavities to promote healing of sinus problems.

### FIELD OF THE INVENTION

The present invention relates to sinus irrigation and, more particularly, to an electric sinus irrigator that is portable and simple, for home or travel use.

### BACKGROUND OF THE INVENTION

More people are having sinus infections or headaches due to allergies, sinusitis or common cold. Taking other types of medication can increase the probability of liver problems and such medications do not always act as quickly as desired. According to many physicians, rinsing out the sinus cavities using saline solution or other forms of liquid medicine is preferable. There is a need for a device that accomplishes this goal and is portable.

Initially there is the neti pot, shaped like a small tea pot, introduced to us and still commonly recommended by doctors for their patients. More recently there is an electric sinus irrigator invented to make the healing liquid to directly contact the sinus cavities more sufficiently, see U.S. Pat. No. 5,899,878.

One problem with the neti pot is that one has to tilt the head to allow the liquid to go in from one nostril and out from the other. It is inconvenient and messy. On the other hand, the aforementioned electric pulse irrigator is too bulky to put in a carry-on bag to travel with, is very noisy and complex and relatively expensive. Also, over time bacteria builds up in some of the components and it is difficult to clean. When turned to the higher stream setting, the pulsing action from the hose actually causes irritation and pain inside the nose.

It is an object of the invention to provide a device to deliver salt water or liquid medicine, herbs, or other agents into all the sinus cavities gently and quietly.

It is another object of the invention to allow said liquid to clean the dirt, allergen and other negative contents out of the sinus cavities.

It is another object of the invention to reduce, maybe heal sinus infections, inflammation, headache or other sinus problems.

It is another object of the invention to provide a portable, simple and affordable electric sinus irrigator for home or travel use.

### SUMMARY OF THE INVENTION

In accordance with the present invention, there is provided a device that has a plastic cup that holds about one cup of liquid connected on top of a base which encloses a mini electric pump. The pump can be powered by standard wall current or by battery. The cord is detachable for portability. There is an on/off switch on the outside of the base. There is also a tube that runs from the outlet of the pump to the spout, which is tapered to allow the top of the spout to fit snugly into the nostril, to deliver the liquid of salt or other agents to the sinus cavities smoothly and quietly for the promotion of healing of sinus problems. The device is a simple unit that fits in the palm of the hand, is portable, convenient, inexpensive and easy to clean.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a left perspective view of an embodiment of a power neti pot (100) according to the present invention which comprising a cylindrical shaped base (1) that is connected to a cup body (5) which also has a lid (7). the cup body (5) has an hourglass shape to allow easy handling. there is a tapered spout (6) which stands on top of one side of the cup body (5), which will fit snugly into a nostril, to deliver the liquid to the sinus cavities without leakage from the same nostril. the base (1) has an electrical on/off switc (3), and a electrical cord with a plug (detachable) (2) for portability. a base cover (4) encloses the base (1) and is also used as the bottom of the cup body (5), which can hold about one cup of liquid. the lid (7) of the cup body (5) is removable for easy cleaning;

FIG. 2 is a top view of an embodiment of a power neti pot (100) according to the present invention which shows the circular shaped top of the cup body (5) with its lid (7) on it. on top of one side of the cup body (5) is a conical shaped tapered spout (6) which is not connected to the lid (7). an outlet tube (9) is held inside the tapered spout (6);

FIG. 3 is a left sectional view of an embodiment of a power neti pot (100) according to the present invention which shows the inside components of the whole unit. at the bottom, enclosed within the base (1), is a mini pump (8) (pulse or non-pulse) and a battery compartment (10). on the side of the base (1) is an electrical on/off switch (3), an electrical cord with a plug (detachable) (2). the base cover (4) has a drain that is recessed into a conical shaped drain cavity, which allows good drainage of the liquid, even when the cup body (5) is tilted at an angle. the intake of the mini pump (8) is connected to this drain as appropriate to allow the mini pump (8) to intake the liquid within the cup body (5). there is also an outlet tube (9) that runs from the outlet of the mini pump (8) to the top of the cup body (5) to the tapered spout (6). there are spout supports (2) (12) connected to the cup body (5) to support the tapered spout (6) from breaking off during use; and

FIG. 4 is a bottom view of an embodiment of a power neti pot (100) according to the present invention which discloses the inside and outside of the base (1). enclosed within the base (1) is the mini pump (8) and the battery compartment (10). on the outside is an electrical on/off switch (3), an electrical cord with a plug (detachable) (2) and an ac/dc switch (11) which allows the user to switch between wall power and battery power. the ac/dc switch (11) is recessed into the bottom of the base (1) such that it does not protrude below the base (1), allowing the unit to stand flat on the bottom of the base (1). the battery compartment (10) can contain two batteries and has a pull out battery compartment door (13) to allow for loading of batteries.

For purposes of clarity and brevity, like elements and components will bear the same designations and numbering throughout the Figures.

### DESCRIPTION OF THE PREFERRED EMBODIMENT

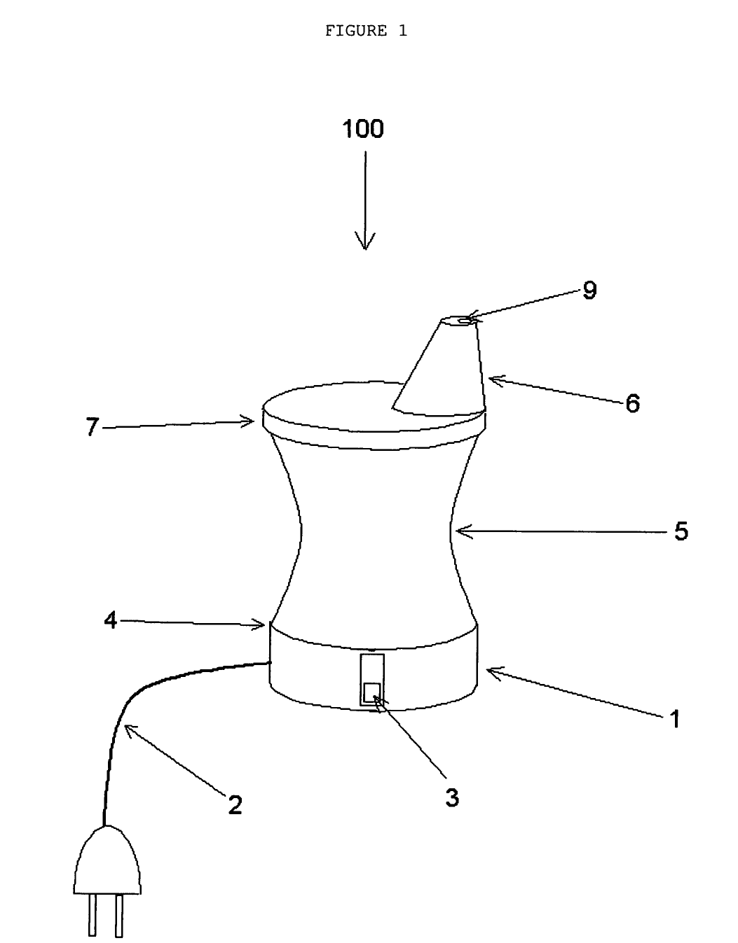

FIG. 1 is a left perspective view of an embodiment of a power neti pot 100 according to the present invention which comprising a cylindrical shaped base 1 that is connected to a cup body 5 which also has a lid 7. The cup body 5 has an hourglass shape to allow easy handling. There is a tapered spout 6 which stands on top of one side of the cup body 5, which will fit snugly into a nostril, to deliver the liquid to the sinus cavities without leakage from the same nostril. The base 1 has an electrical on/off switch 3, and a electrical cord with a plug (detachable) 2 for portability. A base cover 4 encloses the base 1 and is also used as the bottom of the cup body 5, which can hold about one cup of liquid. The lid 7 of the cup body 5 is removable for easy cleaning.

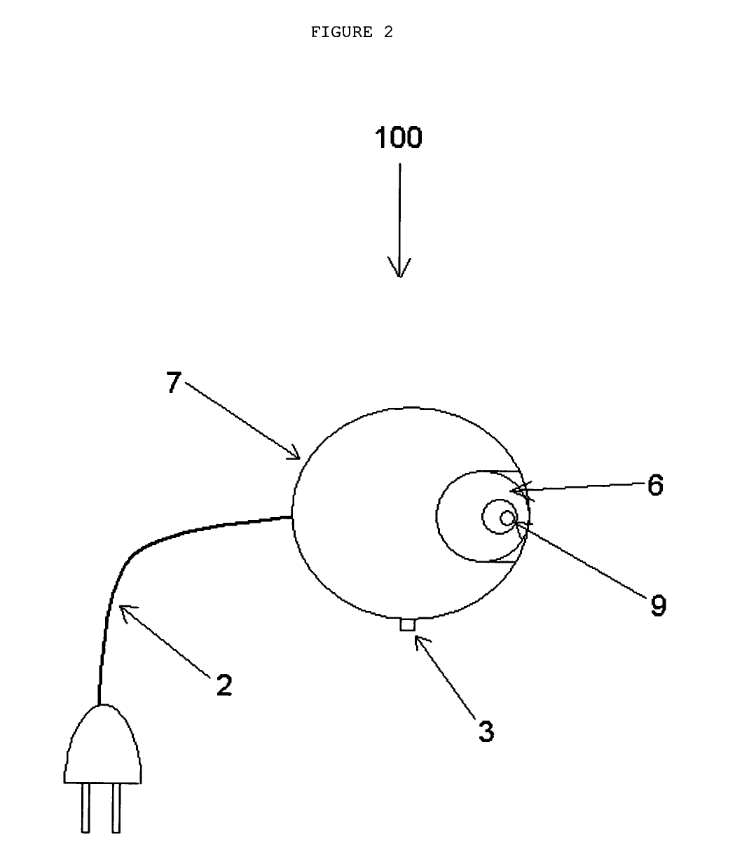

FIG. 2 is a top view of an embodiment of a power neti pot 100 according to the present invention which shows the circular shaped top of the cup body 5 with its lid 7 on it. On top of one side of the cup body 5 is a conical shaped tapered spout 6 which is not connected to the lid 7. An outlet tube 9 is held inside the tapered spout 6.

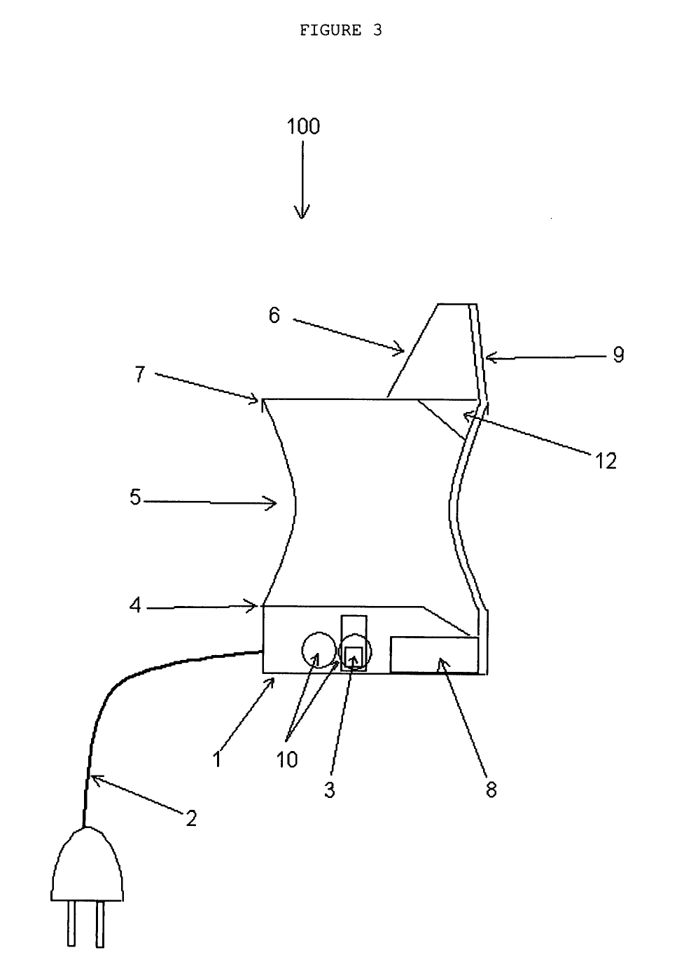

FIG. 3 is a left sectional view of an embodiment of a power neti pot 100 according to the present invention which shows the inside components of the whole unit. At the bottom, enclosed within the base 1, is a mini pump 8 (pulse or non-pulse) and a battery compartment 10. On the side of the base 1 is an electrical on/off switch 3, an electrical cord with a plug (detachable) 2. The base cover 4 has a drain that is recessed into a conical shaped drain cavity, which allows good drainage of the liquid, even when the cup body 5 is tilted at an angle. The intake of the mini pump 8 is connected to this drain as appropriate to allow the mini pump 8 to intake the liquid within the cup body 5. There is also an outlet tube 9 that runs from the outlet of the mini pump 8 to the top of the cup body 5 to the tapered spout 6. There are spout supports (2) 12 connected to the cup body 5 to support the tapered spout 6 from breaking off during use.

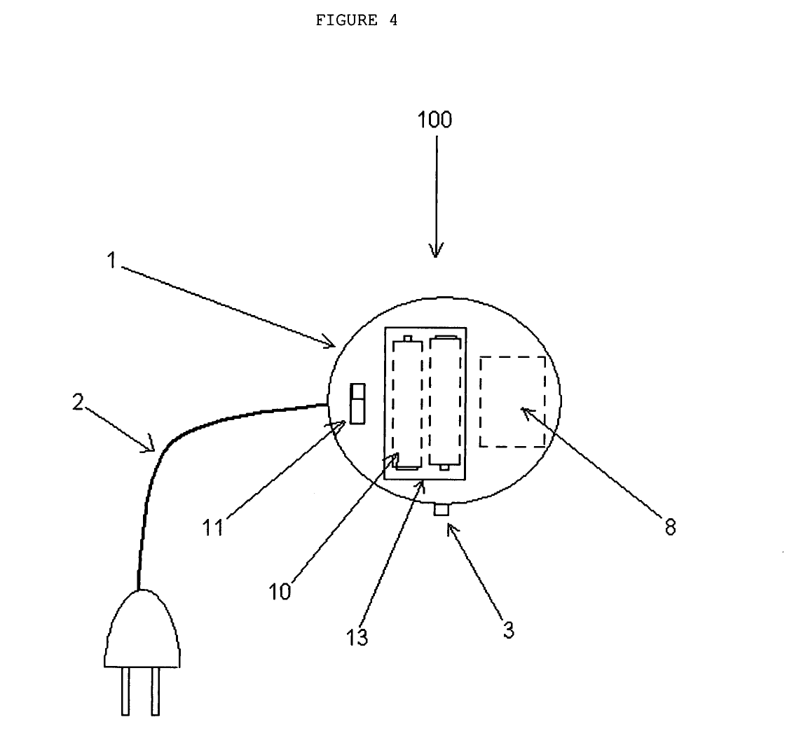

FIG. 4 is a bottom view of an embodiment of a power neti pot 100 according to the present invention which discloses the inside and outside of the base 1. Enclosed within the base 1 is the mini pump 8 and the battery compartment 10. On the outside is an electrical on/off switch 3, an electrical cord with a plug (detachable) 2, and an AC/DC switch 11 which allows the user to switch between wall power and battery power. The AC/DC switch 11 is recessed into the bottom of the base 1 such that it does not protrude below the base 1, allowing the unit to stand flat on the bottom of the base 1. The battery compartment 10 can contain two batteries and has a pull out battery compartment door 13 to allow for loading of batteries.

The majority of the whole unit is made of plastic, except for the metal and other materials (such as wire insulation) required for such components as the switches, cord, plug, the pump and the associated electronic components. The device is one complete unit, hourglass shaped, about 3 to 4 inches in diameter, and about 4 to 5 inches high. The tapered spout 6 is about 2 inches long from top of the cup body 5 to its top.

The unit is in one piece (2 pieces when the cord is detached), is small enough to fit in the palm of a hand, and is light in weight, thus is very portable. It is convenient to use, easy to operate using standard wall current of 110 or 120 volts or battery power, and very affordable.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A portable power neti sinus irrigator for rinsing the sinus cavities with salt water or liquid medicine, herbs or other agents for the promotion of healing of sinus infections, allergies, congestion, headache or other sinus problems as well as rinsing out dirt, allergen and other negative contents, comprising:
means for rinsing the sinus cavaties with salt water or liquid herb or medicine;
means for suporting the cup;
means for turning on or off the power, rigidly connected to said means for suporting the cup;
means for using the wall power, removably connected to said means for suporting the cup;
means for holding the batteries, rigidly connected to said means for suporting the cup;
means for loading the batteries, hingeably connected to said means for holding the batteries;
means for pumping the liquid from the cup to the sinus cavities, rigidly connected to said means for suporting the cup;
means for enclosing the electrical parts in the base and the bottom of the cup, rigidly connected to said means for suporting the cup;
means for holding liquid in the cup, rigidly connected to said means for enclosing the electrical parts in the base and the bottom of the cup;
means for delivering liquid from the pump to the nose, rigidly connected to said means for pumping the liquid from the cup to the sinus cavities;
means for covering the cup, removably connected to said means for holding liquid in the cup;
means for fitting in the nostril snugly, rigidly connected to said means for delivering liquid from the pump to the nose;
means for suporting the tapered spout from breaking during use, rigidly connected to said means for fitting in the nostril snugly, and rigidly connected to said means for holding liquid in the cup; and
means for switching between the wall power and battery power, rigidly connected to said means for suporting the cup.

2. The portable power neti sinus irrigator in accordance with claim 1, wherein said means for rinsing the sinus cavaties with salt water or liquid herb or medicine comprises a power neti pot.

3. The portable power neti sinus irrigator in accordance with claim 1, wherein said means for suporting the cup comprises a base.

4. The portable power neti sinus irrigator in accordance with claim 1, wherein said means for turning on or off the power comprises an electrical on/off switch.

5. The portable power neti sinus irrigator in accordance with claim 1, wherein said means for using the wall power comprises an electrical cord with a plug (detachable).

6. The portable power neti sinus irrigator in accordance with claim 1, wherein said means for holding the batteries comprises a battery compartment.

7. The portable power neti sinus irrigator in accordance with claim 1, wherein said means for loading the batteries comprises a battery compartment door.

8. The portable power neti sinus irrigator in accordance with claim 1, wherein said means for pumping the liquid from the cup to the sinus cavities comprises a mini pump.

9. The portable power neti sinus irrigator in accordance with claim 1, wherein said means for enclosing the electrical parts in the base and the bottom of the cup comprises a base cover.

10. The portable power neti sinus irrigator in accordance with claim 1, wherein said means for holding liquid in the cup comprises a cup body.

11. The portable power neti sinus irrigator in accordance with claim 1, wherein said means for delivering liquid from the pump to the nose comprises an outlet tube.

12. The portable power neti sinus irrigator in accordance with claim 1, wherein said means for covering the cup comprises a lid.

13. The portable power neti sinus irrigator in accordance with claim 1, wherein said means for fitting in the nostril snugly comprises a tapered spout.

14. The portable power neti sinus irrigator in accordance with claim 1, wherein said means for suporting the tapered spout from breaking during use comprises a spout supports (2).

15. The portable power neti sinus irrigator in accordance with claim 1, wherein said means for switching between the wall power and battery power comprises an ac/dc switch.

16. A portable power neti sinus irrigator for rinsing the sinus cavities with salt water or liquid medicine, herbs or other agents for the promotion of healing of sinus infections, allergies, congestion, headache or other sinus problems as well as rinsing out dirt, allergen and other negative contents, comprising:
a power neti pot, for rinsing the sinus cavaties with salt water or liquid herb or medicine;
a base, for suporting the cup;
an electrical on/off switch, for turning on or off the power, rigidly connected to said base;
an electrical cord with a plug (detachable), for using the wall power, removably connected to said base;
a battery compartment, for holding the batteries, rigidly connected to said base;
a battery compartment door, for loading the batteries, hingeably connected to said battery compartment;
a mini pump, for pumping the liquid from the cup to the sinus cavities, rigidly connected to said base;
a base cover, for enclosing the electrical parts in the base and the bottom of the cup, rigidly connected to said base;
a cup body, for holding liquid in the cup, rigidly connected to said base cover;
an outlet tube, for delivering liquid from the pump to the nose, rigidly connected to said mini pump;
a lid, for covering the cup, removably connected to said cup body;
a tapered spout, for fitting in the nostril snugly, rigidly connected to said outlet tube;
a spout supports (2), for suporting the tapered spout from breaking during use, rigidly connected to said tapered spout, and rigidly connected to said cup body; and
an ac/dc switch, for switching between the wall power and battery power, rigidly connected to said base.

17. A portable power neti sinus irrigator for rinsing the sinus cavities with salt water or liquid medicine, herbs or other agents for the promotion of healing of sinus infections, allergies, congestion, headache or other sinus problems as well as rinsing out dirt, allergen and other negative contents, comprising:
a power neti pot, for rinsing the sinus cavaties with salt water or liquid herb or medicine;
a base, for suporting the cup;
an electrical on/off switch, for turning on or off the power, rigidly connected to said base;
an electrical cord with a plug (detachable), for using the wall power, removably connected to said base;
a battery compartment, for holding the batteries, rigidly connected to said base;
a battery compartment door, for loading the batteries, hingeably connected to said battery compartment;
a mini pump, for pumping the liquid from the cup to the sinus cavities, rigidly connected to said base;
a base cover, for enclosing the electrical parts in the base and the bottom of the cup, rigidly connected to said base;
a cup body, for holding liquid in the cup, rigidly connected to said base cover;
an outlet tube, for delivering liquid from the pump to the nose, rigidly connected to said mini pump;
a lid, for covering the cup, removably connected to said cup body;
a tapered spout, for fitting in the nostril snugly, rigidly connected to said outlet tube;
a spout supports (2), for suporting the tapered spout from breaking during use, rigidly connected to said tapered spout, and rigidly connected to said cup body; and
an ac/dc switch, for switching between the wall power and battery power, rigidly connected to said base.
